(19) United States
(12) Patent Application Publication
(10) Pub. No.: US 2024/0176133 A1
(43) Pub. Date: May 30, 2024

(54) Protein Deposition Method For Detecting Protein Removal Effect, Protein Culture Solution and Protein Cleaning and Testing Instrument

(71) Applicant: SUZHOU 3N BIOLOGICAL TECHNOLOGY CO., LTD., Suzhou (CN)

(72) Inventors: Ruichun SUN, Suzhou (CN); Bixia SUN, Suzhou (CN)

(73) Assignee: SUZHOU 3N BIOLOGICAL TECHNOLOGY CO., LTD., Suzhou (CN)

(21) Appl. No.: 18/433,680

(22) Filed: Feb. 6, 2024

Related U.S. Application Data
(63) Continuation of application No. PCT/CN2022/112924, filed on Aug. 17, 2022.

(30) Foreign Application Priority Data
Aug. 20, 2021 (CN) 202110962109.3

(51) Int. Cl. G02B 27/00; B08B 3/08; C11D 7/28; C12N 9/36; G01M 11/02; G02C 7/04

(52) U.S. Cl. CPC G02B 27/0006; B08B 3/08; C11D 7/28; C12N 9/2462; C12Y 302/01017; G01M 11/0257; G02C 7/04

(57) ABSTRACT

A method of protein deposition for detecting a protein removal effect, a protein culture solution, and a protein cleaning and testing instrument, including preparing a protein culture solution: mixing a protein and a deionized water to obtain a diluted protein solution and adjusting a PH value of the diluted protein solution, to obtain the protein culture solution; and culturing the corneal contact lens using the protein culture solution: placing the corneal contact lens and the protein culture solution in a culture container for sealed culture within a culture temperature range for a time duration, to obtain a protein deposited corneal contact lens. The method is more suitable to be used as a testing sample of a protein removal effect testing experiment, reducing the difficulty in testing and judging of the degree of fouling of the corneal contact lens and the cleaning effect of the protein removal effect testing experiment.

### TECHNICAL FIELD

The present disclosure relates to the field of bioprotein detection, and particularly relates to a protein deposition method for detecting a protein removal effect on a corneal contact lens, a protein culture solution, and a cleaning and testing instrument.

### BACKGROUND

The problem of protein deposition on a corneal contact lens has been a concern for the industry for over half a century, triggering a high level of attention to the safety of contact lenses wear in the ophthalmological industry worldwide. In China, due to frequent occurrence of corneal infection caused by the corneal contact lens, the corneal contact lens has been classified as Class III medical devices in 2012 for high risk management. The reasons are as follows: the material structure of contact lenses contains numerous fiber oxygen permeable pores that are invisible to naked eyes. A human eye constantly secretes a large amount of tear. The tear contains a significant amount of dacryolin which is extremely easy to permeate into the fiber oxygen permeable pores to cause reduction (oxygen permeability) of a DK value of the lens, resulting in symptoms such as corneal hypoxia and edema, and also resulting in corneal injury, bacterial infection, corneal inflammation and even visual impairment in severe case.

To effectively remove dacryolin on a surface of the contact lens to guarantee the eye safety of consumers, various methods for removing the dacryolin on the corneal contact lens has been introduced. However, due to the invisibility of the dacryolin to the naked eye and uncertainty about whether they have been completely and effectively eluted and degraded, protein testing methods have been valued and developed gradually, such that whether the dacryolin is completely degraded is effectively verified through scientific detection. However, at present, a testing method for effective protein removal of the corneal contact lens on the market is limited due to a special structure of criss-crossed oxygen permeable pores in the corneal contact lens, and it is not possible to find a method capable of directly, effectively and accurately measuring a specific quantity value of the dacryolin adsorbed by a hard corneal contact lens. Therefore, the detection result of the method is not comprehensive, only having certain reference significance, and cannot be taken as a professional detection ground for quantitatively and qualitatively testing the degradation degree of proteins.

To verify the dacryolin removal effect on the corneal contact lens, at present, researchers usually prepare an artificial tear based on a human eye environment, culture the corneal contact lens with the artificial tear a for a period of time, quantize the amount of the proteins adsorbed by the corneal contact lens according to a difference in proteins in the artificial tear before and after culturing, and then clean the cultured corneal contact lens for protein removal. This experimental approach is reasonable. However, to culture the corneal contact lens with a conventional artificial tear, the proteins adsorbed to the lens are movable and are unlikely to deposit. Some of the proteins on the cultured corneal contact lenses can be removed by only soaking in liquid (normal saline, a care solution). It interferes with actual effective verification of protein removal. Moreover, this method is only suitable for testing the protein concentration with a precision testing instrument, lacking the capability for intuitive observation based on visual phenomena. Therefore, experimental results are less convincing to the general audience, and it is more difficult to test and determine the degree of fouling and the cleaning effect on the lens by the testing experiment.

Thus, it is a present need of a novel technical solution to solve the above problems.

### SUMMARY

At present, proteins adsorbed to a corneal contact lens cultured with a conventional artificial tear are usually movable and are unlikely to form deposits on the corneal contact lens in a culture process. Therefore, a removal effect on the proteins on the corneal contact lens cultured with the conventional artificial tear cannot be observed visually in a protein removal effect testing experiment, which makes it is more difficult to test and judge the degree of fouling and the cleaning effect on the corneal contact lens by the testing experiment. An object of the present disclosure is to solve the problems in the current technology. In one aspect, the present disclosure provides a protein deposition method for detecting a protein removal effect on a corneal contact lens, which adopts the following technical solution:

A protein deposition method on a corneal contact lens, comprises: preparing a protein culture solution: mixing a protein and a deionized water to obtain a diluted protein solution and adjusting a PH value of the diluted protein solution to obtain the protein culture solution; and culturing the corneal contact lens by using the protein culture solution: placing the corneal contact lens and the protein culture solution in a culture container for sealed culture within a predetermined culture temperature range for a predetermined time duration, to obtain a protein deposited corneal contact lens.

In the above technical solution, further, the preparing a protein culture solution specifically comprises: mixing one or a combination of more than one of lysozyme, bovine serum albumin (BSA), or globulin, with the deionized water to obtain the diluted protein solution, and mixing the diluted protein solution with one of an acidic solution or an alkaline solution to adjust the PH value of the diluted protein solution until the PH value of the diluted protein solution reaches an isoelectric point of the protein in the diluted protein solution, to obtain the protein culture solution, wherein a protein concentration of the protein culture solution is no more than 6.0 mg/ml

Further, the protein concentration of the protein culture solution is no more than 6.0 mg/ml.

Further, the higher the culture temperature is, the higher the deposition rate of the proteins on the cultured corneal contact lens is, and the more proteins are deposited.

Further, the culture temperature is no more than 42° C.

Further, the predetermined culture temperature range is from 36° C. to 42° C.

Further, a hard corneal contact lens and the protein culture solution are placed in the culture container for culturing at a constant temperature for the predetermined time duration, to obtain the protein deposited hard corneal contact lens.

Further, in a lens culture process, the lens culture process is divided into a first stage, a second stage and a third stage in sequential order according to a growth rate of the amount of proteins deposited on the corneal contact lens; when the corneal contact lens is cultured in the first stage, an average protein deposition rate on the corneal contact lens is marked as V1, when the corneal contact lens is cultured in the second stage, the average protein deposition rate on the corneal contact lens is marked as V2, and when the corneal contact lens is cultured in the third stage, the average protein deposition rate on the corneal contact lens is marked as V3, wherein V1>V2>V3 and V3 approaches to zero.

Further, the method further comprises: placing a NaCl solution in the diluted protein solution or the protein culture solution, to obtain the protein culture solution with an osmotic pressure, wherein the protein culture solution with an osmotic pressure that maintains an inherent form of a soft corneal contact lens.

Further, the concentration of the NaCl solution is 0.9%.

Further, the osmotic pressure of the protein culture solution with the osmotic pressure ranges from 260 to 340 mOsm/kgH₂O.

Further, the lysozyme, the BSA, the globulin and the deionized water are mixed, to obtain the diluted protein solution, a protein concentration of the diluted protein solution being 2.2 mg/ml, wherein a concentration of the lysozyme is 1.9 mg/ml, a concentration of the BSA is 0.2 mg/ml, and a concentration of the globulin is 0.1 mg/ml; or, the lysozyme and the deionized water are mixed, to obtain the diluted protein solution, the protein concentration of the diluted protein solution being 2.2 mg/ml.

Further, the lysozyme is extracted from egg white, the isoelectric point of the lysozyme is 10.88, and a NaOH solution or an HCl solution is placed in the diluted protein solution until the PH value of the diluted protein solution is 10.88, to obtain the protein culture solution.

In another aspect, the present disclosure further provides a protein culture solution for a corneal contact lens. The protein culture solution comprises a protein, a deionized water, and one of an acidic solution or an alkaline solution, wherein the protein, the deionized water, and one of the acidic solution or the alkaline solution are mixed to form the protein culture solution, and a PH value of the protein culture solution is an isoelectric point of the protein in the protein culture solution.

In the above technical solution, further, the protein comprises one or more of lysozyme, BSA and globulin; the acidic solution comprises an HCl solution that decreases the PH value of the protein culture solution; and the alkaline solution comprises a NaOH solution that increases the PH value of the protein culture solution.

In yet another aspect, the present disclosure further provides another protein culture solution for a corneal contact lens, comprising the protein culture solution for a corneal contact lens and further comprising a NaCl solution, wherein the protein culture solution for a corneal contact lens and the NaCl solution are mixed, to form a protein culture solution with an osmotic pressure, and the protein culture solution with an osmotic pressure that maintains an inherent form of a soft corneal contact lens.

Further, the osmotic pressure of the protein culture solution with an osmotic pressure ranges from 260 to 340 mOsm/kgH₂O.

Further, the concentration of the NaCl solution is 0.9%.

In another aspect, the present disclosure provides a method for detecting a protein removal effect on a corneal contact lens, comprising: culturing the corneal contact lens using a protein culture solution, to obtain a corneal contact lens having observable protein deposition on its surface according to the protein deposition method on a corneal contact lens according to claim 1; photographing the corneal contact lens having observable protein deposition on its surface, to obtain a lens image before cleaning; cleaning the corneal contact lens by a corneal contact lens cleaning method, and photographing the cleaned corneal contact lens, to obtain a cleaned lens image; and comparing and observing the cleaned lens image and the lens image before cleaning, to obtain a protein removal effect of the corneal contact lens cleaning method

In the above technical solution, further, the method further comprises: after completing the culturing the corneal contact lens, testing a residual protein concentration in the protein culture solution, and calculating to obtain an original protein content on the corneal contact lens according to following equation: an original protein content on the corneal contact lens=(the original protein concentration in the protein culture solution−the residual protein concentration in the protein culture solution)×volume of the protein culture solution−a deposited protein content in a petri dish; performing a protein extraction on the deposited protein in the petri dish in which the lens culture has been completed through a trifluoroacetic acid solution, testing the protein concentration in the trifluoroacetic acid solution after completing the protein extraction, and calculating to obtain the deposited protein content in the petri dish according to the volume of the trifluoroacetic acid solution; cleaning the cultured corneal contact lens by the corneal contact lens cleaning method; performing a protein extraction on the cleaned corneal contact lens through a trifluoroacetic acid solution, testing the protein concentration in the trifluoroacetic acid solution after completing the protein extraction, and calculating to obtain the residual protein content on the cleaned contact lens according to the volume of the trifluoroacetic acid solution; and calculating to obtain a protein removal rate of the corneal contact lens cleaning method through the original protein content on the corneal contact lens and the residual protein content on the cleaned contact lens.

Based on the protein culture solution for a corneal contact lens provided above, the present disclosure provides a protein cleaning and testing instrument, comprising: a body, having an accommodating cavity containing a power storage device, a circuit board and a switching assembly, wherein the power storage device and the switching assembly are electrically connected to the circuit board, respectively; an electrophoretic dissociation chamber is arranged on one side of the body and comprises two electrophoretic dissociation grooves; each of the electrophoretic dissociation grooves is internally provided with at least two electrophoretic dissociation probes; each electrophoretic dissociation groove is filled with an electrolyte solution containing Cl⁻; the electrophoretic dissociation probes are in contact with the electrolyte solution containing Cl⁻; and the electrophoretic dissociation probes are electrically connected to the circuit board; and a cleaning and testing chamber being provided with two cleaning and testing grooves, the cleaning and testing grooves being filled with a trifluoroacetic acid solution.

In the above technical solution, further, the cleaning and testing chamber is arranged on the side, provided with the electrophoretic dissociation chamber, of the body, and the electrophoretic dissociation chamber and the cleaning and testing chamber are oppositely arranged in a spaced manner.

Further, the two electrophoretic dissociation probes in the electrophoretic dissociation chamber form a positive electrode and a negative electrode in a circuit loop, a dacryolin attached to a surface of the corneal contact lens to be cleaned is charged in the electrolyte solution containing CI, and the charged dacryolin moves toward the electrode with electrical property opposite thereto.

Further, Cl⁻ in the electrolyte solution moves towards the positive electrode, loses an electron and is oxidized to a chlorine; the chlorine is dissolved in the electrolyte solution to generate a hypochlorous acid; the hypochlorous acid and the dacryolin are subjected to an oxidizing reaction in the electrophoretic dissociation grooves to degrade the dacryolin.

Further, the corneal contact lens cleaned in the electrophoretic dissociation chamber is accommodated in the cleaning and testing chamber for cleaning again, and the protein content of a cleaning solution in the cleaning and testing chamber is tested to obtain a protein cleaning effect of the electrophoretic dissociation chamber.

Further, when the corneal contact lens is a hard corneal contact lens, the cleaning and testing groove is filled with a 0.2% trifluoroacetic acid solution as the cleaning solution; when the corneal contact lens is a soft corneal contact lens, the cleaning solution filling the cleaning and testing groove comprises 50 parts of acetonitrile, 50 parts of pure water and 0.2 part of 100% trifluoroacetic acid.

Further, the corneal contact lens to be cleaned is obtained by culturing in the protein culture solution or is obtained by being worn by a human eye.

Further, the corneal contact lens is soaked and cleaned in the cleaning and testing groove filled with the cleaning solution.

Further, each of the cleaning and testing grooves in the cleaning and testing chamber comprises a liquid holding groove and a hollowed groove arranged in the liquid holding groove, the liquid holding groove comprises a transduction sheet and a plastic seal assembly fixing the transduction sheet, a gap is reserved between the transduction sheet and an outer wall of the hollowed groove, and a conductive wire of the transduction sheet is connected to the circuit board.

Further, the trifluoroacetic acid solution as the cleaning solution and the corneal contact lens to be cleaned are accommodated in the cleaning and testing groove, the transduction sheet is powered on to form a micro high speed vortex having a cleaning effect on the dacryolin on the surface of the corneal contact lens in the cleaning and testing groove, and the dacryolin on the surface of the corneal contact lens is separated from the lens and becomes free in the cleaning solution.

Further, each of the cleaning and testing grooves is covered with a sealing cover.

Compared with the current technology, the present disclosure at least has the following one or more beneficial effects:
1. The present disclosure provides a protein deposition method on a corneal contact lens. Use of the protein deposition method can guarantee that a lot of proteins in the protein culture solution are deposited on the corneal contact lens, and the amount of proteins attached to the lens is improved, forming stains on the transparent lens, and thus, the degree of fouling of the lens may be more visually observed by naked eyes. Moreover, the proteins deposited on the lens according to the protein deposition method provided by the present disclosure are unlikely to be washed and removed by normal saline, a multi-functional care solution and the like. The lens cultured by such method is more suitable to be used as a testing sample for the protein removal effect testing experiment, which reduces the difficulty in testing and judging of the degree of fouling and the cleaning effect on the lens by the protein removal effect testing experiment.
2. By culturing the lens with the protein deposition method provided by the present disclosure, the deposition rate of the proteins on the lens can be improved, and the amount of the proteins can be increased. Compared with a conventional method to culture the lens, the efficiency of the method is higher, and the protein removal effect of the lens with a great deposition amount of the proteins can be more easily observed visually by naked eyes, so that the judging difficulty of experiments is reduced, the testing cost is lowered, and the dependence on the testing instrument is reduced.
3. Due to the presence of ionized side chains on surfaces of proteins in the conventional artificial tear, the proteins carry net charges, and therefore, there are mutual repulsive force between molecules of the proteins in the conventional artificial tear. By using the conventional artificial tear to culture the corneal contact lens, the proteins adsorbed to the lens are movable and are unlikely to deposit. Some of the proteins can be removed by only soaking in liquid (normal saline, a care solution), which naturally interferes with the actual effect verification of protein removal. The pH value of the protein in the protein culture solution provided by the present disclosure is just at its isoelectric point, and the proteins carry no charge, so that the repulsive force between the molecules of the proteins can be reduced. At a certain temperature (appropriate near 37° ° C. for lens culture), the proteins may be deposited to the surface of the corneal contact lens slowly. Such deposited proteins can be observed with naked eyes and optical devices, are unlikely to wash and remove with the normal saline and multi-functional care solution and the like, and can be recovered and extracted.
4. The amount of proteins on the corneal contact lens cultured by the conventional artificial tear is small, the proteins are unstable and prone to detachment from the lens in small quantities as the lens is soaked in the conventional liquid. Moreover, as for the protein removal effect testing completed by the lens cultured by the conventional artificial tear, it is only suitable for testing the protein concentration with instruments and the effect cannot be directly observed from appearance phenomenon, which increases the experimental difficulty in testing and judging of the degree of fouling and the cleaning effect on the lens. The amount of proteins deposited on the lens cultured by the protein culture solution provided by the present disclosure is larger, the degree of fouling of protein attachment cab be seen with naked eyes. Therefore, the cleaning effect can be directly judged by observing the appearance with naked eyes in testing the protein removal effect by using the lens cultured by the protein culture solution provided by the present disclosure, making it more convincing to the general audience.
5. The protein culture solution provided by the present disclosure can be used to culture both the hard corneal contact lens and the soft corneal contact lens. The 0.9% NaCl solution when added into the protein culture solution can adjust the osmotic pressure of the protein culture solution, so as to maintain the inherent form of the soft corneal contact lens.
6. Based on the protein culture solution and the protein deposition method on a corneal contact lens provided by the present disclosure, the present disclosure further provides a method for detecting a protein removal effect on the corneal contact lens. The method cultures the lens to be cleaned and tested by applying the protein culture solution and the protein deposition method on a corneal contact lens. By taking the cultured lens as the testing sample, the testing result can be visually criticized through apparent clarity, which reduces the difficulty in testing and judging of the cleaning effect.

### BRIEF DESCRIPTION OF THE DRAWINGS

To describe the technical solutions in the embodiments of the present disclosure or in the current technology more clearly, the drawings required for describing the embodiments or existing technologies are briefly described below. Apparently, the drawings in the following description are merely some embodiments of the present disclosure, and a person of ordinary skill in the art may still derive other drawings from these drawings without making creative efforts.

FIG. 1 depicts a culture condition of a hard corneal contact lens after culturing the hard corneal contact lens by a protein culture solution for 4 hours in Embodiment 3;

FIG. 2 depicts a culture condition of a hard corneal contact lens after culturing the hard corneal contact lens by a protein culture solution for 12 hours in Embodiment 3;

FIG. 3 depicts a culture condition of a soft corneal contact lens after culturing the soft corneal contact lens by a protein culture solution for 4 hours in Embodiment 4;

FIG. 4 depicts culture condition of a soft corneal contact lens after culturing the soft corneal contact lens by a protein culture solution for 12 hours in Embodiment 4; and

FIG. 5 is a structural exploded view of a protein cleaning and testing instrument in Embodiment 8.

In the drawings: 100-body; 110-upper cover of body; 120-base of body; 130-accommodating cavity; 140-circuit board; 200-electrophoretic dissociation chamber; 210-electrophoretic dissociation groove; 220-electrophoretic dissociation probe; 230-electrode connector of electrophoretic dissociation probe; 240-upper cover of electrophoretic dissociation chamber; 250-base of electrophoretic dissociation chamber; 260-sealing cover; 300-cleaning and testing chamber; and 310-cleaning and testing groove.

### DETAILED DESCRIPTION

The technical solution of the present disclosure will be clearly and completely described below with reference to the embodiments of the present disclosure. Apparently, the described examples are merely a part of, rather than, all of the embodiments of the present disclosure. All other embodiments obtained by a person of ordinary skill in the art based on the disclosed embodiments without creative efforts shall fall within the protection scope of the present disclosure.

In the description of the present disclosure, it shall be understood that unless explicitly specified and limited, a person of ordinary skill in the art may understand the specific meanings of the above terms in the present disclosure according to specific situations.

The gist of the present disclosure is further described below with reference to embodiments.

### Embodiment 1

When a corneal contact lens is cultured by using a conventional artificial tear, proteins adsorbed to the lens are movable and are unlikely to deposit. Some of the proteins can be removed by only soaking in liquid (normal saline, a care solution), which interferes with the actual effect verification of protein removal. Moreover, this method is only suitable for testing the protein concentration with instruments, and the effect cannot be directly observed from appearance phenomenon, which increases the experimental difficulty in testing and judging of the degree of fouling and the cleaning effect on the lens.

To solve the problems in the current technology, the present disclosure provides a protein culture solution that is easy to form protein deposits on the corneal contact lens. The protein culture solution is mainly different from the artificial tear in that the protein in the protein culture solution do not carry net charges. Due to the presence of ionized side chains on surfaces of molecules of proteins in the conventional artificial tear, the molecules of proteins carry net charges (that is, the surfaces of the proteins carry charges that are not uncanceled, with like charges repelling each other and unlike charges attracting each other). Therefore, the molecules of the proteins in the artificial tear are unlikely to deposit due to charge interaction. Moreover, the stability of the molecules of the proteins attached to the surface of the lens is poor. The proteins are movable on the surface of the lens and are likely to be separated from the lens when being soaked in the liquid. According to the protein culture solution provided by the present disclosure, by titrating the ionized side chains on the surfaces of the proteins (the ionized side chains on the surfaces of the proteins are titrated by adjusting the PH value of the culture solution, so that the PH value of the culture solution reaches the isoelectric point of the proteins containing therein), realizing that the PH environment where the proteins are located is just the isoelectric point thereof. The net charges on the surfaces of the proteins are zero, and thus there is no repulsion caused by charge action between the molecules of the proteins in the culture solution. Therefore, the proteins are easier to deposit and are more stable, and the deposited proteins are also more difficult to detach from the lens even being soaked in liquid.

Additional information to the isoelectric point of the molecules of the proteins is given below:

Due to the presence of the ionized side chains on the surfaces of the proteins, the proteins carry net charges. Because all the side chains are titratable, for each protein, there is such a pH value which makes its surface net charge be zero, i.e., an isoelectric point.

At the isoelectric point, the molecules of the proteins exist in the form of zwitter-ions, with molecular net charges being zero (that is, positive and negative charges are equal). In this case, the action force between molecules of protein molecular particles is weakened because there is no mutual repulsion of same charges in the solution, and the particles are extremely likely to collide and agglomerate to generate deposits. Therefore, at the isoelectric point, the proteins have the minimum solubility, and thus are most likely to form deposits. Many physical properties at the isoelectric point such as viscosity, expansibility and osmotic pressure will be decreased, facilitating the filtering of a suspension. All other pH values outside the isoelectric point are separated and purified by electrophoresis and ion-exchange column chromatography according to the net charges carried by the proteins.

By applying the isoelectric point of the proteins, the present disclosure provides a protein culture solution for a corneal contact lens. The protein culture solution comprises the components such as proteins, deionized water, and one of an acidic solution or an alkaline solution. The proteins, deionized water and one of acidic solution or alkaline solution are mixed to form the protein culture solution. The PH value of the protein culture solution is the isoelectric point of the protein in the protein culture solution.

In one embodiment, the proteins for preparing the protein culture solution may be one or more of lysozyme, BSA and globulin, which is also a protein configuration with reference to the main protein components of the dacryolin in the human eye. After the proteins are dissolved with the deionized water, the PH value of the solution is confirmed. The PH value of the protein solution is adjusted to the isoelectric point of the proteins with an HCl solution and/or a NaOH solution. The HCl solution as the acidic solution may decrease the PH value of the protein culture solution. The NaOH solution as the alkaline solution may increase the PH value of the protein culture solution.

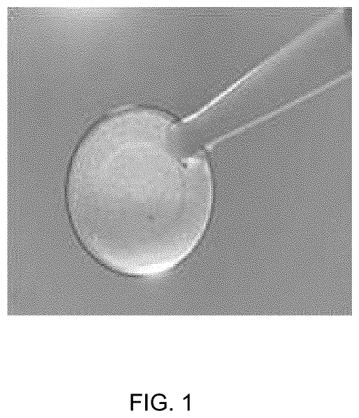

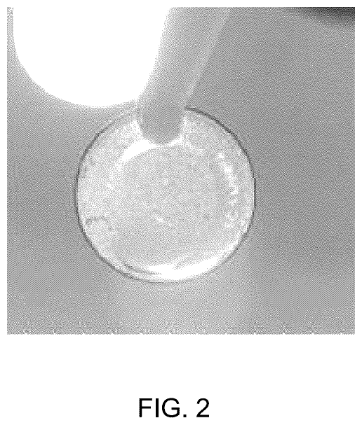

In one embodiment, 22 mg by weight of lysozyme may be weighed. 20 ml of deionized water is added to fully dissolve the lysozyme. A NaOH solution or an HCl solution is added to the dissolved solution for pH adjustment, so that the pH value of the final solution is near the isoelectric point 10.88 of the lysozyme (the lysozyme is extracted from egg white, and the pH values needs to be determined according to the isoelectric point of the proteins in the formulated solution) to obtain the protein culture solution (the lysozyme solution) with the concentration of 2.2 mg/ml. A hard corneal contact lens is cultured at a constant temperature of 37° C. with the protein culture solution provided in tis embodiment. An image shown in FIG. 1 is obtained after culturing for 4 h. FIG. 1 exhibits the image of the hard corneal contact lens cultured with the protein culture solution for 4 hours shown in this embodiment. It can be clearly seen that the lens is turbid and opaque, with white particles attached thereto. If the hard corneal contact lens is continuously cultured for 12 h, an image shown in FIG. 2 may be obtained. Compared with FIG. 1, more white particles are gathered on the hard corneal contact lens in FIG. 2.

Therefore, the hard corneal contact lens shown in FIG. 1 and FIG. 2 verifies that the protein culture solution provided by the present disclosure can indeed culture the lens with a relatively large amount of proteins deposited.

### Embodiment 2

The protein culture solution provided by the above embodiment may be used to culture either the hard corneal contact lens (hard lens for short) or a soft corneal contact lens (soft lens for short). However, by culturing the soft corneal contact lens with the protein culture solution provided by the above embodiment may result in deformation of the soft lens due to an osmotic pressure. However, with respect to lens culture for testing, the protein culture solution provided by the above embodiment may also be used to culture the soft corneal contact lens.

In the protein removal testing experiment, if it is needed to keep the inherent form of the soft corneal contact lens (soft lens for short), the protein culture solution provided by this embodiment may be used.

The protein culture solution provided by this embodiment is a protein culture solution obtained by adding the NaCl solution in the protein culture solution provided by Embodiment 1 and can maintain the osmotic pressure of the soft lens. The protein culture solution as a result of addition of the NaCl solution. The osmotic pressure range is 260-340 mOsm/kgH₂O. The NaCl solution may be a 0.9% NaCl solution, also known as normal saline. The PH value of the NaCl solution is neutral, which does not affect the isoelectric point of the protein culture solution.

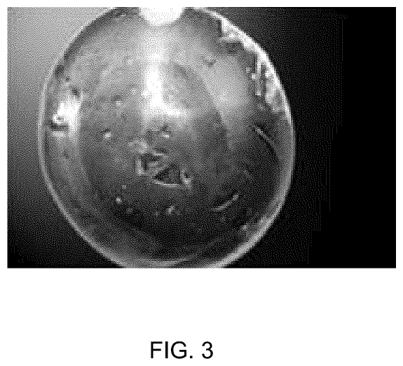

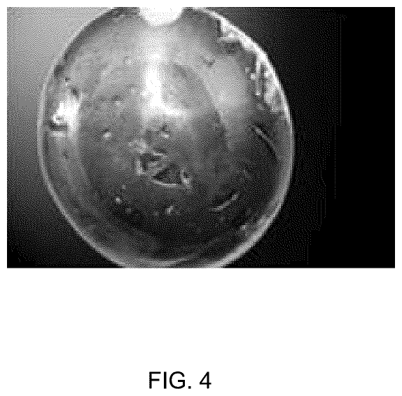

In one embodiment, 22 mg by weight of lysozyme and 90 mg by weight of sodium chloride are weighed. 10 ml of deionized water is added to fully dissolve the lysozyme and sodium chloride. A NaOH solution or an HCl solution is added to the dissolved solution for pH adjustment, so that the pH value of the final solution is near the isoelectric point 10.88 of the lysozyme and the osmotic pressure of the final solution is 260-340 mOsm/kgH₂O, to obtain the protein culture solution (the lysozyme solution) with the concentration of 2.2 mg/ml. The soft corneal contact lens is cultured at a constant temperature of 37° C. with the protein culture solution provided in this embodiment. An image shown in FIG. 3 is obtained after culturing for 4 h. FIG. 3 exhibits the image of the soft corneal contact lens cultured with the protein culture solution for 4 hours shown in this embodiment. It can be clearly seen that the lens is turbid and opaque, with white particles uniformly attached thereto. If the soft corneal contact lens is continuously cultured for 12 h, an image shown in FIG. 4 may be obtained. Compared with FIG. 3, more white particles are gathered on the soft corneal contact lens in FIG. 4, and the lens is blurrier. The soft corneal contact lens for culturing in this embodiment is a CooperVision Biomedics Now® monthly disposable contact lens.

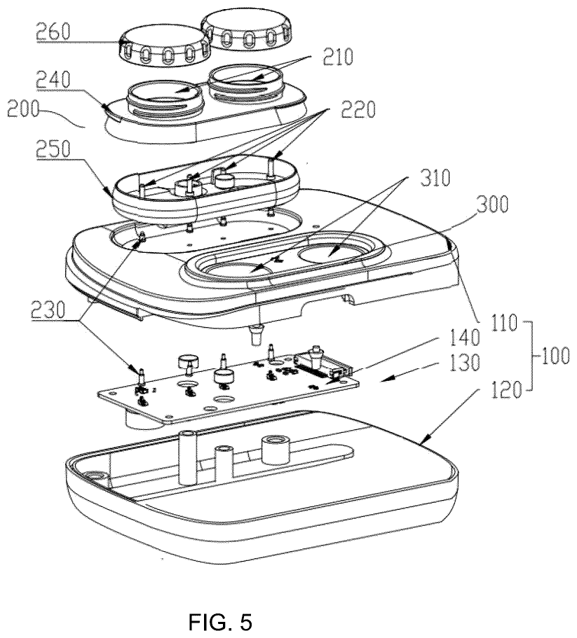

It can be seen from FIG. 4 and FIG. 5 that the protein culture solution with the normal saline can maintain the inherent form of the soft lens, so that deformation of the soft lens is avoided.

It still needs to emphasize that the normal saline (0.9% sodium chloride solution) itself has a certain protein removal effect. When the proteins are cultured on the hard corneal contact lens, research may be carried out directly without the normal saline. However, if there is a need to maintain the inherent form of the soft corneal contact lens, the osmotic pressure range (the osmotic pressure is 260-340 mOsm/kgH₂O) of the lens shall be guaranteed when the proteins are cultured. Otherwise, the soft lens is likely to deform. Therefore, when the protein solution for the soft lens is prepared, it needs to add 9 mg/ml NaCl.

The main protein component in the protein culture solution provided by the present disclosure comprises lysozyme, BSA and globulin, which mainly the components simulating dacryolin of the human eye. At present, there is still no uniform report stating the ratio of the artificial tear. The protein culture solution in this embodiment of the present disclosure is prepared according to the proportion of (1.9 mg/ml) lysozyme, (0.2 mg/ml) BSA and (0.1 mg/ml) globulin. The total concentration of proteins is 2.2 mg/ml. To facilitate quantitative testing, the solution is prepared based on lysozyme. Therefore, the isoelectric point is selected as 10.88 (the isoelectric point of the lysozyme extracted from egg white, different from the isoelectric point of the lysozyme extracted from a human body which is 6.99). Different proteins have different isoelectric points. For the protein component based on BSA, the isoelectric point of the solution may be selected as 4.6.

### Embodiment 3

This embodiment provides a protein deposition method on a corneal contact lens. According to the method, the proteins can be deposited on a surface of the corneal contact lens, and the deposition form on the surface of the lens can be directly observed. The protein adsorbed deposits on the lens are unlikely to be dissolved and removed by soaking in liquid for a long time. Therefore, whether the deposited proteins adsorbed by the lens are removed and the removal energy efficiency can be effectively proved.

The protein deposition method on a corneal contact lens provided in this embodiment comprises the following steps:
Step 1: preparing a protein culture solution: mixing a protein and a deionized water to obtain a diluted protein solution and adjusting a PH value of the diluted protein solution to obtain the protein culture solution; and
Step 2: culturing the corneal contact lens by using the protein culture solution: placing the corneal contact lens and the protein culture solution in a culture container for sealed culture within a predetermined culture temperature range for a predetermined time duration, to obtain a protein deposited corneal contact lens.

The preparing a protein culture solution in Step 1 specifically comprises:
Step 11: mixing one or a combination of more than one of lysozyme, bovine serum albumin (BSA), or globulin, with the deionized water to obtain the diluted protein solution; and
Step 12: mixing the diluted protein solution with one of an acidic solution or an alkaline solution to adjust the PH value of the diluted protein solution until the PH value of the diluted protein solution reaches an isoelectric point of the protein in the diluted protein solution, to obtain the protein culture solution.

It is found by the applicant(s) through a large number of experiments that the protein concentration of the protein culture solution is no more than 6.0 mg/ml, and if the protein concentration of the protein culture solution is too high, the lens will be more fragile when taken out from the protein culture solution. Of course, the larger the protein concentration of the protein culture solution is, the higher the degree of attachment of the proteins is, which is more beneficial for experimental testing. However, when the testing instrument is precise, the protein culture solution with a relatively low concentration may also be used to culture the lens as long as it is capable of being tested by the instrument and the experiment is completed in an assisted manner.

Further, it is found by the applicant(s) through a large number of experiments that at the isoelectric point, the culture rate of proteins on the lens of the protein culture solution is related to the culture temperature. The higher the culture temperature is, the higher the deposition rate of the cultured proteins on the corneal contact lens is, and the more proteins are deposited. The culture temperature is no more than 42° C. At a temperature exceeding 42° C., the proteins are likely to degenerate and may not be recovered for quantitative determination.

A preferred culture temperature range is from 36° C.-42° C.

In one embodiment, in Step 2, the hard corneal contact lens and the protein culture solution are added into a culture container for culturing at a constant temperature for the predetermined time duration, to obtain a protein deposited hard corneal contact lens. The longer the culture time is, the greater the amount of proteins deposited on the cultured corneal contact lens is. The amount of proteins deposited is related to the protein content in the protein culture solution. When the amount of proteins deposited reaches the maximum value of the amount of proteins deposited, with the extension of the culture time, the amount of proteins deposited on the corneal contact lens tends to be constant. The lens culture process may also be divided into a first stage, a second stage and a third stage in sequential order according to a growth rate of the amount of proteins deposited on the corneal contact lens. When the corneal contact lens is cultured in the first stage, an average protein deposition rate on the corneal contact lens is marked as V1, when the corneal contact lens is cultured in the second stage, the average protein deposition rate on the corneal contact lens is marked as V2, and when the corneal contact lens is cultured in the third stage, the average protein deposition rate on the corneal contact lens is marked as V3, where V1>V2>V3 and V3 approaches to zero. With the extension of the culture time, the growth rate of the amount of proteins deposited on the corneal contact lens is smaller. If a rectangular coordinate system is established by taking the amount of proteins deposited on the lens as a vertical axis and time as a horizontal axis to draw a broken line graph representing the deposition rate of the proteins on the lens, it may be found that the broken line graph is divided into three sections, and with the passage of time, the slope of a broken line is gradually decreased until it approaches to 0. The above protein culture solution is the protein culture solution provided in Embodiment 1.

In one embodiment, the 2.2 mg/ml protein culture solution may be prepared by taking lysozyme, BSA and globulin as protein components. The concentration of the lysozyme is 1.9 mg/ml, the concentration of the BSA is 0.2 mg/ml, and the concentration of the globulin is 0.1 mg/ml.

In another embodiment, the 2.2 mg/ml protein culture solution may also be prepared by mixing the lysozyme as the protein component and the deionized water.

In one embodiment, the hard corneal contact lens may be cultured by the protein deposition method on a corneal contact lens. The method comprises the following steps:
preparing a protein culture solution: 22 mg of lysozyme and 20 ml of deionized water are mixed, to obtain a diluted protein solution with the protein concentration of 2.2 mg/ml (the lysozyme is extracted from egg white, and the isoelectric point of the lysozyme is 10.88); and a NaOH solution or an HCl solution is added into the diluted protein solution until the PH value of the diluted protein solution is 10.88, to obtain the protein culture solution;
culturing the hard corneal contact lens by using the obtained protein culture solution: the cleaned hard corneal contact lens is placed in a sealable culture container, then the protein culture solution prepared above is added as needed, and finally, sealed culture is carried out. The hard corneal contact lens is a Euclid hard permeable contact lens for shaping a cornea.

Because the deposition rates of the proteins at different environment temperatures are different, the higher the temperature is, the higher the deposition rate of the proteins is (the temperature is no more than 42° C.). By simulating a 37° C. constant temperature environment of the human eye for culture, the adsorption rate of the lysozyme deposits is much higher than culture at a normal temperature. Moreover, the longer the culture time is, the more the proteins deposited are, and the time may be selected according to an actual demand (of course, it is also related to the total amount of the proteins, and the extension of the time after the maximum amount of deposition is achieved will not make the amount of deposition increased).

After culturing for 4 hours according to the culture method, an image of the culture condition of the hard lens shown in FIG. 1 is obtained. After further culturing for 12 h, an image of the culture condition of the hard lens shown in FIG. 2 is obtained. It can be seen that according to the protein culture solution and the protein culture method provided by the present disclosure, a lot of proteins can be indeed deposited on the lens, which contributes to comparative verification of the protein removal testing experiment.

### Embodiment 4

This embodiment provides a protein deposition method on a corneal contact lens based on Embodiment 3. This method differs from the method provided in Embodiment 3 in that: in the protein deposition method on a corneal contact lens provided by this embodiment, a NaCl solution may be added in Step 11 or Step 12 in Embodiment 3. That is, the NaCl solution is added into the diluted protein solution or the protein culture solution, to obtain the protein culture solution with an osmotic pressure (the protein culture solution in the protein deposition method on a corneal contact lens provided in Embodiment 3 does not have an osmotic pressure that maintains the inherent form of the soft lens). The protein culture solution with an osmotic pressure that maintains the inherent form of the soft corneal contact lens. The above protein culture solution provided in Embodiment 2 is obtained.

In one embodiment, the concentration of the added NaCl solution is 0.9%, i.e., normal saline. After the normal saline is added, the osmotic pressure range of the protein culture solution is 260-340 mOsm/kgH₂O.

In one embodiment, the soft corneal contact lens may be cultured by the protein deposition method on a corneal contact lens in this embodiment. The method comprises the following steps:
preparing a protein culture solution: 22 mg of lysozyme, 90 mg of sodium chloride and 10 ml of deionized water are mixed, to obtain a diluted protein solution with the protein concentration of 2.2 mg/ml (the lysozyme is extracted from egg white, and the isoelectric point of the lysozyme is 10.88); and a NaOH solution or an HCl solution is placed in the diluted protein solution until the PH value of the diluted protein solution is 10.88 and the osmotic pressure of the solution is kept at 260-340 mOsm/kgH₂O, to obtain the protein culture solution;

Culturing the soft corneal contact lens by using the obtained protein culture solution: the cleaned soft corneal contact lens is placed in a sealable culture container, then the protein culture solution prepared above is added as needed, and finally, sealed culture is carried out. The soft corneal contact lens is a CooperVision Biomedics Now® monthly disposable contact lens.

Because the deposition rates of the proteins at different environment temperatures are different, the higher the temperature is, the higher the deposition rate of the proteins is (the temperature is no more than 42° C.). By simulating a 37° C. constant temperature environment of the human eye for culture, the adsorption rate of the lysozyme deposits is much higher than culture at a normal temperature. Moreover, the longer the culture time is, the more the proteins deposited are, and the time may be selected according to an actual demand (of course, it is also related to the total amount of the proteins, and the extension of the time after the maximum amount of deposition is achieved will not make the amount of deposition increased).

After culturing for 4 hours according to the culture method, an image of the culture condition of the soft lens shown in FIG. 3 is obtained. After further culturing for 12 h, an image of the culture condition of the soft lens shown in FIG. 4 is obtained. It can be seen that according to the protein culture solution and the protein culture method provided by the present disclosure, a lot of proteins can be indeed deposited on the lens, which contributes to comparative verification of the protein removal testing experiment, and the protein culture solution added with the NaCl solution may indeed maintain the inherent form of the soft lens.

### Embodiment 5

To verify that it is indeed difficult to remove part of proteins from the lens cultured by the protein culture solution provided by the present disclosure by soaking in liquid, this embodiment provides a verification experiment.

A cultured corneal contact lens is taken out from a protein culture solution, and was photographed for storage; and the corneal contact lens is washed and soaked in normal saline or a standard salt solution and is photographed for storage after a certain period of time, and images are compared visually to obtain the protein removal of the lens by the normal saline or standard salt solution.

It is found by the applicant(s) through multiple repeated experiments that the protein deposits on the lens cannot be completely removed after the lens is washed and soaked in the normal saline or standard salt solution. The proteins on the lens may be observed with naked eyes or through an optical instrument, and the protein content may also be measured after the proteins are sufficiently extracted by an ultrasonic method with 0.2% trifluoroacetic acid.

### Embodiment 6

Based on the protein deposition method on a corneal contact lens provided in Embodiment 3 or 4, this embodiment provides a method for detecting a protein removal effect on a corneal contact lens. The method comprises the following steps:
a: culturing the corneal contact lens using a protein culture solution according to the protein deposition method on a corneal contact lens provided in Embodiment 3 or 4, to obtain a corneal contact lens having observable protein deposition on its surface;
b: photographing the corneal contact lens having observable protein deposition on its surface, to obtain a lens image before cleaning;
c: cleaning the corneal contact lens by a corneal contact lens cleaning method, and photographing the cleaned corneal contact lens, to obtain a cleaned lens image; and
d: comparing and observing the cleaned lens image and the lens image before cleaning, to obtain a protein removal effect of the corneal contact lens cleaning method.

The corneal contact lens cleaning method in Step c comprises various cleaning methods on the market, for example, normal saline soaking and cleaning, care solution soaking and cleaning, ultrasonic cleaning, electrophoretic cleaning and electrophoretic dissociation cleaning. All the methods for testing the protein removal effect on the corneal contact lens in this embodiment may be used to verify the cleaning effects of the various cleaning methods.

The method for testing the protein cleaning effect provided in this embodiment is more intuitive. A user may directly observe the protein removal with naked eyes. The method cultures the lens to be cleaned and tested by applying the protein culture solution and the protein deposition method on a corneal contact lens. By taking the cultured lens as the testing sample, the testing result may be visually criticized through apparent clarity, which reduces the difficulty in judging and testing of the cleaning effect is reduced, and makes the user more convincing in the testing result.

### Embodiment 7

Based on the protein deposition method on a corneal contact lens provided in Embodiment 3 or 4, this embodiment provides another method for detecting a protein removal effect on a corneal contact lens. The method comprises the following steps:
A: culturing the corneal contact lens using the protein culture solution according to the protein deposition method on a corneal contact lens provided in Embodiment 3 or 4, to obtain a corneal contact lens having observable protein deposition on its surface;
B: after completing the culturing the corneal contact lens, testing a residual protein concentration in the protein culture solution, and calculating to obtain an original protein content on the corneal contact lens according to following equation:: an original protein content on the corneal contact lens=(the original protein concentration in the protein culture solution−the residual protein concentration in the protein culture solution)×volume of the protein culture solution−a deposited protein content in a petri dish;
C: performing a protein extraction on the deposited protein in the petri dish in which the lens culture has been completed through a trifluoroacetic acid solution, testing the protein concentration in the trifluoroacetic acid solution after completing the protein extraction, and calculating to obtain the deposited protein content in the petri dish according to the volume of the trifluoroacetic acid solution;
D: cleaning the cultured corneal contact lens by the corneal contact lens cleaning method;
E: performing a protein extraction on the cleaned corneal contact lens through a trifluoroacetic acid solution, testing the protein concentration in the trifluoroacetic acid solution after completing the protein extraction, and calculating to obtain the residual protein content on the cleaned contact lens according to the volume of the trifluoroacetic acid solution; and
F: calculating to obtain a protein removal rate of the corneal contact lens cleaning method through the original protein content on the corneal contact lens and the residual protein content on the cleaned contact lens.

“The corneal contact lens cleaning method” in Step C comprises various cleaning methods on the market, for example, normal saline soaking and cleaning, care solution soaking and cleaning, ultrasonic cleaning, electrophoretic cleaning and electrophoretic dissociation cleaning. All the methods for testing the protein removal effect on the corneal contact lens in this embodiment may be used to verify the cleaning effects of the various cleaning methods.

In the method for testing the protein cleaning effect provided in this embodiment, the lens is cultured with the protein culture solution provided by the present disclosure. The protein deposition may be directly observed with naked eyes. That is, the amount of deposition of proteins is high, and the amount of testing samples is high. In the protein removal testing experiment, the accuracy of the experimental result is improved.

### Embodiment 8

Based on the protein culture solution for a corneal contact lens provided above, the present disclosure provides a protein cleaning and testing instrument. Referring to FIG. 5, the protein cleaning and testing instrument comprises a body 100, having an accommodating cavity 130 containing a power storage device, a circuit board 140 and a switching assembly. The power storage device and the switching assembly are electrically connected to the circuit board 140, respectively. An electrophoretic dissociation chamber 200 is arranged on one side of the body 100. The electrophoretic dissociation chamber 200 comprises two electrophoretic dissociation grooves 210. Each of the electrophoretic dissociation grooves 210 is internally provided with at least two electrophoretic dissociation probes 220. The electrophoretic dissociation groove 210 is filled with an electrolyte solution containing Cl⁻. The electrophoretic dissociation probes 220 are in contact with the electrolyte solution containing Cl⁻. The electrophoretic dissociation probes 220 are electrically connected to the circuit board 140. The protein cleaning and testing instrument further comprises a cleaning and testing chamber 300. Two cleaning and testing grooves 310 are arranged in the cleaning and testing chamber 300. The cleaning and testing grooves 310 are filled with a trifluoroacetic acid solution.

The protein cleaning and testing instrument provided in the above technical solution may be used to clean and test proteins. The lens is cultured with the protein culture solution provided by the present disclosure, to obtain a lens to be cleaned. The lens to be cleaned is placed in the electrophoretic dissociation chamber 200 for protein removal and cleaning. Then the cleaned lens is placed in the cleaning and testing groove 310 to extract the proteins. The residual protein amount of the lens subjected to electrophoretic dissociation cleaning is tested. Thus, cleaning and testing of the proteins can be achieved.

Provided below is a protein cleaning and testing instrument for realizing cleaning and testing of protein in conjunction with FIG. 5, comprising a body 100, having an accommodating cavity 130 containing a power storage device, a circuit board 140 and a switching assembly. The power storage device and the switching assembly are electrically connected to the circuit board 140, respectively. An electrophoretic dissociation chamber 200 is arranged on one side of the body 100. The electrophoretic dissociation chamber 200 comprises two electrophoretic dissociation grooves 210. Each of the electrophoretic dissociation grooves 210 is internally provided with at least one group of electrophoretic dissociation probes 220. The electrophoretic dissociation groove 210 is filled with an electrolyte solution containing CI. The electrophoretic dissociation probes 220 are in contact with the electrolyte solution containing Cl⁻. The electrophoretic dissociation probes 220 are electrically connected to the circuit board 140. The protein cleaning and testing instrument further comprises a cleaning and testing chamber 300. Two cleaning and testing grooves 310 are arranged in the cleaning and testing chamber 300. The cleaning and testing grooves 310 are filled with a trifluoroacetic acid solution. The cleaning and testing chamber 300 is arranged on the side, provided with the electrophoretic dissociation chamber 200, of the body 100. The electrophoretic dissociation chamber 200 and the cleaning and testing chamber 300 are oppositely arranged in a spaced manner.

When the electrophoretic dissociation chamber 200 is powered on, the two electrophoretic dissociation probes 220 in the electrophoretic dissociation chamber 200 form a positive electrode and a negative electrode in a circuit loop. Dacryolin attached to a surface of the corneal contact lens to be cleaned is charged in the electrolyte solution containing Cl⁻, and the charged dacryolin moves toward an electrode position with electrical property opposite thereto, generating a protein electrophoretic phenomenon. Further, Cl⁻ in the electrolyte solution moves towards the positive electrode, loses an electron and is oxidized to chlorine. The chlorine is dissolved in the electrolyte solution to generate hypochlorous acid. The hypochlorous acid and the dacryolin are subjected to an oxidizing reaction in the electrophoretic dissociation grooves 210 of the lens. The dacryolin is degraded. That is, an electrolysis phenomenon occurs in the electrophoretic dissociation grooves 210 to generate the hypochlorous acid with a protein degradation effect.

When it is needed to test the protein cleaning effect, the corneal contact lens cleaned in the electrophoretic dissociation chamber 200 is placed in the cleaning and testing chamber 300 for cleaning again. The protein content of a cleaning solution (the cleaning solution plays a role of extracting the proteins on the lens) in the cleaning and testing chamber 300 is tested to obtain a protein cleaning effect of the electrophoretic dissociation chamber 200.

When the corneal contact lens is a hard corneal contact lens, the cleaning and testing groove 310 is filled with a 0.2% trifluoroacetic acid solution as the cleaning solution. When the corneal contact lens is a soft corneal contact lens, the cleaning solution filling the cleaning and testing groove 310 comprises 50 parts of acetonitrile, 50 parts of pure water and 0.2 part of 100% trifluoroacetic acid.

In one embodiment, to observe the protein removal effect visually with naked eyes, the protein culture solution provided by the present disclosure may be used to culture the corneal contact lens. Of course, the lens to be cleaned may also be worn by the human eye. However, in this way, the protein content on the lens is insufficiently observed by the human eye, and protein removal testing needs to be completed by virtue of a protein content testing instrument.

In one embodiment, the corneal contact lens is soaked and cleaned in the testing and cleaning groove filled with the cleaning solution, that is, it is left stand and soaked with the trifluoroacetic acid solution. In comparison, the longer the soaking time is, the better the protein extraction effect is. After a period of time, the protein content in the trifluoroacetic acid solution may be tested, i.e., the residual protein amount on the lens cleaned in the electrophoretic dissociation chamber 200.

In one embodiment, the cleaning and testing chamber 300 in the present disclosure may also have a vibrating cleaning function. When the proteins are extracted with the trifluoroacetic acid solution, with vibratory incubation extraction, the extraction rate is higher. If the lens is left stand and soaked, the extraction rate will be lower. In order to accelerate the testing process, vibratory protein extraction may be selected. Each of the cleaning and testing grooves is provided with a sealing cover (not shown). The sealing cover plays a role of sealing the cleaning and testing groove. When the proteins are extracted in the cleaning and testing groove, the sealing cover seals the cleaning and testing groove.

Therefore, this embodiment provides a cleaning and testing chamber 300 for a protein cleaning and testing instrument. Each of the cleaning and testing grooves in the cleaning and testing chamber 300 comprises a liquid holding groove and a hollowed groove arranged in the liquid holding groove. The liquid holding groove comprises a transduction sheet and a plastic seal assembly fixing the transduction sheet. A gap is reserved between the transduction sheet and an outer wall of the hollowed groove. A conductive wire of the transduction sheet is connected to the circuit board 140. The trifluoroacetic acid solution as the cleaning solution and the corneal contact lens to be cleaned are accommodated in the cleaning and testing groove 310. The transduction sheet is powered on to form a micro high speed vortex having a cleaning effect on the dacryolin on the surface of the corneal contact lens in the cleaning and testing groove 310. The dacryolin on the surface of the corneal contact lens is separated from the lens and becomes free in the cleaning solution. Referring to FIG. 5, the protein cleaning and testing instrument shown in FIG. 5 has the electrophoretic dissociation chamber 200 and the cleaning and testing chamber 300. The cleaning and testing chamber 300 may comprise two cleaning and testing grooves 310 soaking and extracting the proteins and may also provide an environment for vibratory protein extraction to the cleaning and testing grooves 310. The cleaning and testing grooves 310 shown in FIG. 5 do not have a function of vibratory protein extraction, but can extract the proteins by way of standing and soaking, which, for the lens, means that the proteins on the surface of the lens are removed.

In conclusion, the present disclosure provides a method of protein deposition, a protein culture solution and a cleaning and testing instrument for detecting a protein removal effect on a corneal contact lens. Use of the protein deposition method can guarantee that proteins in the protein culture solution are deposited heavily on the corneal contact lens, increasing the amount of protein adhering to the lens surface, forming stains on the transparent lens, and thus, the degree of fouling of the lens may be more visually observed by naked eyes. Moreover, the proteins deposited on the lens according to the protein deposition method provided by the present disclosure are unlikely to be washed and removed by normal saline, a multi-functional care solution, etc. The lens cultured by such method is more suitable as a testing sample for the protein removal effect testing experiment, which reduces the difficulty in testing and judging of the degree of fouling and the cleaning effect on the lens by the protein removal effect testing experiment.

In the description of this specification, the description of the reference terms “an embodiment”, “some embodiments”, “an example”, “a specific example”, “some examples”, and the like means that specific features, structures, materials or characteristics described in combination with the embodiment(s) or example(s) are comprised in at least one embodiment or example of the present disclosure. In this specification, schematic descriptions of the above terms are not necessarily directed at the same embodiment or example. In addition, the described specific features, structures, materials, or characteristics may be combined in a proper manner in any one or more of the embodiments or examples. In addition, those skilled in the art may combine different embodiments or examples described in this specification.

Although the embodiments of the present disclosure have been shown and described above, it may be understood that the above embodiments are exemplary and shall not be understood as limitation to the present disclosure. A person of ordinary skill in the art may make changes, modifications, replacements, and variations to the above embodiments within the scope of the present disclosure.

What is claimed is:

1. A method of protein deposition on a corneal contact lens, the method comprising:
preparing a protein culture solution comprising: mixing a protein and a deionized water to obtain a diluted protein solution and adjusting a PH value of the diluted protein solution to obtain the protein culture solution; and
culturing the corneal contact lens by using the protein culture solution comprising: placing the corneal contact lens and the protein culture solution in a culture container for sealed culture within a predetermined culture temperature range for a predetermined time duration, to obtain a protein deposited corneal contact lens.

2. The method according to claim 1, wherein the preparing a protein culture solution further comprises:
mixing one or a combination of more than one of lysozyme, bovine serum albumin (BSA), or globulin, with the deionized water to obtain the diluted protein solution, and mixing the diluted protein solution with one of an acidic solution or an alkaline solution to adjust the PH value of the diluted protein solution until the PH value of the diluted protein solution reaches an isoelectric point of the protein in the diluted protein solution, to obtain the protein culture solution, wherein a protein concentration of the protein culture solution is no more than 6.0 mg/ml.

3. The method according to claim 1, wherein higher a culture temperature is, higher a deposition rate of the proteins on the cultured corneal contact lens is, and more proteins are deposited; and the culture temperature is no more than 42° C.

4. The method according to claim 1, wherein
the predetermined culture temperature range is from 36° C. to 42° C.;
a hard corneal contact lens and the protein culture solution are placed in the culture container for culturing at a constant temperature for the predetermined time duration, to obtain the protein deposited hard corneal contact lens; and
in a lens culture process, the lens culture process is divided into a first stage, a second stage and a third stage in sequential order according to a growth rate of the amount of proteins deposited on the corneal contact lens; when the corneal contact lens is cultured in the first stage, an average protein deposition rate on the corneal contact lens is marked as V1, when the corneal contact lens is cultured in the second stage, the average protein deposition rate on the corneal contact lens is marked as V2, and when the corneal contact lens is cultured in the third stage, the average protein deposition rate on the corneal contact lens is marked as V3, wherein V1>V2>V3 and V3 approaches to zero.

5. The method according to claim 1, further comprising:
adding a NaCl solution into one of the diluted protein solution or the protein culture solution, to obtain the protein culture solution with an osmotic pressure, wherein the protein culture solution with an osmotic pressure that maintains an inherent form of a soft corneal contact lens,
a concentration of the NaCl solution is 0.9%; and
the osmotic pressure of the protein culture solution with the osmotic pressure ranges from 260 to 340 mOsm/kgH₂O.

6. The method according to claim 2, further comprising:
mixing the lysozyme, the BSA, the globulin and the deionized water, to obtain the diluted protein solution, a protein concentration of the diluted protein solution being 2.2 mg/ml, wherein a concentration of the lysozyme is 1.9 mg/ml, a concentration of the BSA is 0.2 mg/ml, and a concentration of the globulin is 0.1 mg/ml; or,
mixing the lysozyme and the deionized water, to obtain the diluted protein solution, the protein concentration of the diluted protein solution being 2.2 mg/ml;
wherein the lysozyme is extracted from egg white, the isoelectric point of the lysozyme is 10.88, and a NaOH solution or an HCl solution is added into the diluted protein solution until the PH value of the diluted protein solution is 10.88, to obtain the protein culture solution.

7. A protein culture solution for a corneal contact lens, comprising a protein, a deionized water, and one of an acidic solution or an alkaline solution,
wherein the protein, the deionized water, and one of the acidic solution or the alkaline solution are mixed to form the protein culture solution, and a PH value of the protein culture solution is an isoelectric point of the protein in the protein culture solution.

8. The solution according to claim 7, wherein the protein comprises one or more of lysozyme, BSA, or globulin;
the acidic solution comprises an HCl solution that decreases the PH value of the protein culture solution; and
the alkaline solution comprises a NaOH solution that increases the PH value of the protein culture solution.

9. The solution according to claim 7, further comprising a NaCl solution,
wherein the protein culture solution for a corneal contact lens and the NaCl solution are mixed, to form a protein culture solution with an osmotic pressure, and the protein culture solution with an osmotic pressure that maintains an inherent form of a soft corneal contact lens;
the osmotic pressure of the protein culture solution with an osmotic pressure ranges from 260 to 340 mOsm/kgH₂O; and
the concentration of the NaCl solution is 0.9%.

10. A method for detecting a protein removal effect on a corneal contact lens, comprising:
culturing the corneal contact lens using a protein culture solution, to obtain a corneal contact lens having observable protein deposition on its surface according to the protein deposition method on a corneal contact lens according to claim 1;
photographing the corneal contact lens having observable protein deposition on its surface, to obtain a lens image before cleaning;
cleaning the corneal contact lens by a corneal contact lens cleaning method, and photographing the cleaned corneal contact lens, to obtain a cleaned lens image; and
comparing and observing the cleaned lens image and the lens image before cleaning, to obtain a protein removal effect of the corneal contact lens cleaning method.

11. The method according to claim 10, further comprising:
after completing the culturing the corneal contact lens, testing a residual protein concentration in the protein culture solution, and calculating to obtain an original protein content on the corneal contact lens according to following equation: an original protein content on the corneal contact lens=(the original protein concentration in the protein culture solution−the residual protein concentration in the protein culture solution)×volume of the protein culture solution−a deposited protein content in a petri dish;
performing a protein extraction on the deposited protein in the petri dish in which the lens culture has been completed through a trifluoroacetic acid solution, testing the protein concentration in the trifluoroacetic acid solution after completing the protein extraction, and calculating to obtain the deposited protein content in the petri dish according to the volume of the trifluoroacetic acid solution;
cleaning the cultured corneal contact lens by the corneal contact lens cleaning method;
performing a protein extraction on the cleaned corneal contact lens through the trifluoroacetic acid solution, testing the protein concentration in the trifluoroacetic acid solution after completing the protein extraction, and calculating to obtain the residual protein content on the cleaned contact lens according to the volume of the trifluoroacetic acid solution; and
calculating to obtain a protein removal rate of the corneal contact lens cleaning method through the original protein content on the corneal contact lens and the residual protein content on the cleaned contact lens.

12. A protein cleaning and testing instrument, comprising:
a body, having an accommodating cavity containing a power storage device, a circuit board and a switching assembly, wherein the power storage device and the switching assembly are electrically connected to the circuit board, respectively; an electrophoretic dissociation chamber is arranged on one side of the body and comprises two electrophoretic dissociation grooves; each of the electrophoretic dissociation grooves is internally provided with at least two electrophoretic dissociation probes; each electrophoretic dissociation groove is filled with an electrolyte solution containing Cl⁻; the electrophoretic dissociation probes are in contact with the electrolyte solution containing Cl⁻; and the electrophoretic dissociation probes are electrically connected to the circuit board; and
a cleaning and testing chamber being provided with two cleaning and testing grooves, the cleaning and testing grooves being filled with a trifluoroacetic acid solution.

13. The instrument according to claim 12, wherein
the cleaning and testing chamber is arranged on the side, provided with the electrophoretic dissociation chamber, of the body, and the electrophoretic dissociation chamber and the cleaning and testing chamber are oppositely arranged in a spaced manner;
the two electrophoretic dissociation probes in the electrophoretic dissociation chamber form a positive electrode and a negative electrode in a circuit loop, a dacryolin attached to a surface of the corneal contact lens to be cleaned is charged in the electrolyte solution containing Cl⁻, and the charged dacryolin moves toward the electrode with electrical property opposite thereto;
Cl⁻ in the electrolyte solution moves towards the positive electrode, loses an electron and is oxidized to a chlorine; the chlorine is dissolved in the electrolyte solution to generate a hypochlorous acid; the hypochlorous acid and the dacryolin are subjected to an oxidizing reaction in the electrophoretic dissociation grooves to degrade the dacryolin;
the corneal contact lens cleaned in the electrophoretic dissociation chamber is accommodated in the cleaning and testing chamber for cleaning again, and the protein content of a cleaning solution in the cleaning and testing chamber is tested to obtain a protein cleaning effect of the electrophoretic dissociation chamber;
when the corneal contact lens is a hard corneal contact lens, the cleaning and testing groove is filled with a 0.2% trifluoroacetic acid solution as the cleaning solution; when the corneal contact lens is a soft corneal contact lens, the cleaning solution filling the cleaning and testing groove comprises 50 parts of acetonitrile, 50 parts of pure water and 0.2 part of 100% trifluoroacetic acid;
the corneal contact lens to be cleaned is obtained by culturing in the protein culture solution or is obtained by being worn by a human eye; and
the corneal contact lens is soaked and cleaned in the cleaning and testing groove filled with the cleaning solution.

14. The instrument according to claim 12, wherein
each of the cleaning and testing grooves in the cleaning and testing chamber comprises a liquid holding groove and a hollowed groove arranged in the liquid holding groove, the liquid holding groove comprises a transduction sheet and a plastic seal assembly fixing the transduction sheet, a gap is reserved between the transduction sheet and an outer wall of the hollowed groove, and a conductive wire of the transduction sheet is connected to the circuit board;
the trifluoroacetic acid solution as the cleaning solution and the corneal contact lens to be cleaned are accommodated in the cleaning and testing groove, the transduction sheet is powered on to form a micro high speed vortex having a cleaning effect on the dacryolin on the surface of the corneal contact lens in the cleaning and testing groove, and the dacryolin on the surface of the corneal contact lens is separated from the lens and becomes free in the cleaning solution; and
each of the cleaning and testing grooves is covered with a sealing cover.
